(19) United States
(12) Patent Application Publication
(10) Pub. No.: US 2025/0161573 A1
(43) Pub. Date: May 22, 2025

(54) Injection Device And Methods Of Use Therefor

(71) Applicant: United States Government As Represented By The Department Of Veterans Affairs, Washington, DC (US)

(72) Inventors: Curtis Lee Lowery, III, Houston, TX (US); Curtis Lee Lowery, JR., Honolulu, HI (US)

(21) Appl. No.: 18/953,726

(22) Filed: Nov. 20, 2024

Related U.S. Application Data
(60) Provisional application No. 63/600,965, filed on Nov. 20, 2023.

(51) Int. Cl. A61M 5/315; A61M 5/20

(52) U.S. Cl. CPC A61M 5/31511; A61M 5/2033; A61M 2205/33

(57) ABSTRACT

An apparatus configured to dispense medication from a cartridge is disclosed. The apparatus includes a housing that is configured to couple to the cartridge. A piston is movable within the housing along a path of travel from a first position to a second position. A spring biases the piston toward the second position. A catch is configured to retain the piston in the first position. A trigger is configured to release the catch to permit the spring to move the piston to the second position. A sensor is configured to detect movement of the piston from the first position. A controller is in electrical communication with the sensor. The controller is configured to initiate a timer upon receiving a signal from the sensor indicative of movement of the piston from the first position.

### CROSS-REFERENCE TO RELATED APPLICATIONS

This application claims priority to U.S. Provisional Patent Application No. 63/600,965, filed Nov. 20, 2023, the entirety of which is hereby incorporated by reference herein.

### FIELD

This disclosure relates to injectors for injecting medication into individuals.

### BACKGROUND

In various circumstances and for various reasons it can be beneficial to inject a medication into an individual and to then know how recently the medication was injected. However, conventionally, medical professionals rely on, when available, estimates from memories of those present.

Further, at risk individuals can benefit from N-Acetylcysteine (NAC) upon having a heart attack, strokes, battlefield blood loss-related ischemic/reperfusion injuries, or high-dose radiation exposure. However, NAC is conventionally provided by a medical professional once the individual has arrived at a medical facility, even though earlier provision would have been desirable.

### SUMMARY

Described herein, in various aspects, is an apparatus configured to dispense medication from a cartridge. The apparatus includes a housing that is configured to couple to the cartridge. A piston is movable within the housing along a path of travel from a first position to a second position. A spring biases the piston toward the second position. A catch is configured to retain the piston in the first position. A trigger is configured to release the catch to permit the spring to move the piston to the second position. A sensor is configured to detect movement of the piston from the first position. A controller is in electrical communication with the sensor. The controller is configured to initiate a timer upon receiving a signal from the sensor indicative of movement of the piston from the first position (e.g., as the piston is released by the trigger and moves to the second position to deliver the medication), thereby allowing for accurate tracking of the timing of medication delivery.

Also disclosed is an autoinjector including apparatus configured to dispense medication from a cartridge, and a cartridge coupled to the housing. The cartridge includes an enclosure configured to couple to the housing. A syringe is movable within the enclosure from a stowed position to a deployed position, the syringe containing a medication. The syringe includes a needle. When the syringe is in the stowed position, the needle is within the enclosure. When the syringe is in the deployed position, the needle projects from the enclosure. The syringe includes a plunger that is configured to engage the piston as the piston moves from the first position to the second position to effect flow of the medication through the needle.

Also disclosed is an autoinjector including a housing, a piston movable within the housing along a path of travel from a first position to a second position, and a spring biasing the piston toward the second position. A catch that is configured to retain the piston in the first piston. A trigger that is configured to release the catch to permit the spring to move the piston to the second position. A cartridge is coupled to the housing, the cartridge includes an enclosure configured to couple to the housing, a syringe movable within the enclosure from a stowed position to a deployed position, the syringe containing a medication. The syringe includes a needle. When the syringe is in the stowed position, the needle is within the enclosure, and when the syringe is in the deployed position, the needle projects from the enclosure. The syringe comprises a plunger that is configured to engage the piston as the piston moves from the first position to the second position to effect flow of the medication through the needle. The enclosure defines a stop that is configured to engage the syringe when the syringe is at the deployed position. The cartridge comprises a ring that is configured to engage the piston. The ring is movable within the enclosure to push the syringe toward the deployed position. The ring and the piston cooperate to define a first threshold force above which the piston passes through the ring. Upon contact between the piston and the ring, the piston is configured to apply to the ring a force below the threshold force so that the piston pushes against the ring to push the syringe to the second position until the syringe engages the stop. Upon the syringe contacting the stop, the syringe is configured to apply a force above the threshold force to drive the syringe through the ring and contact the plunger.

Also disclosed herein is a cartridge configured to couple to the housing of the apparatus that is configured dispense medication. The cartridge includes an enclosure configured to couple to the housing of the apparatus. A syringe is movable within the enclosure from a stowed position to a deployed position, the syringe containing a medication. The syringe includes a needle. When the syringe is in the stowed position, the needle is within the enclosure, and when the syringe is in the deployed position, the needle projects from the enclosure. The syringe includes a plunger that is configured to engage a piston to effect flow of the medication through the needle. The enclosure defines a stop that is configured to engage the syringe when the syringe is at the deployed position. The cartridge comprises a ring that is configured to engage the piston. The ring is movable within the enclosure to push the syringe toward the deployed position. The ring and the piston cooperate to define a first threshold force above which the piston passes through the ring. Upon contact between the piston and the ring, the piston is configured to apply to the ring a force below the threshold force so that the piston pushes against the ring to push the syringe to the second position until the syringe engages the stop. Upon the syringe contacting the stop, the syringe is configured to apply a force above the threshold force to drive the syringe through the ring and contact the plunger.

A method can comprise injecting medication into a subject that has been suspected as having a heart attack, a stroke, a large dose of radiation exposure, or loss of blood, or other event causing an increase in free radical exposure. The medication can comprise N-Acetylcysteine (NAC).

Additional advantages of the disclosed systems and methods will be set forth in part in the description that follows, and in part will be obvious from the description, or may be learned by practice of the claimed invention. The advantages of the disclosed systems and methods will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a perspective view of an exemplary injector as disclosed herein, wherein the autoinjector is in a stowed configuration before deployment.

FIG. 2 is a perspective view of the exemplary injector of FIG. 1 at an intermediate configuration after deployment.

FIG. 3 is a perspective of the exemplary injector of FIG. 1 in a used configuration after dispensing medication.

FIG. 4 is a perspective view of the exemplary injector of FIG. 1, in which the cartridge is separated from the deployment apparatus.

FIG. 5 is a perspective view showing reloading of the spring of the deployment apparatus of the exemplary injector of FIG. 1.

FIG. 6 the deployment apparatus of the exemplary injector of FIG. 1 once the deployment apparatus has been reloaded.

FIG. 7 is a cross sectional view of the exemplary injector of FIG. 1 in the stowed configuration.

FIG. 8 is a cross sectional view of the exemplary injector of FIG. 1 in the used configuration.

FIG. 9A shows a partial cross sectional view of the exemplary injector of FIG. 1 with the piston in a retained configuration. FIG. 9B shows a partial cross sectional view of the exemplary injector of FIG. 1 once the trigger has been actuated to release the piston from the retained configuration. FIG. 9C shows a partial cross sectional view of the exemplary injector of FIG. 1 as the piston is moving through the injector.

FIG. 10 shows another exemplary injector as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

It must be noted that as used herein and in the appended claims, the singular forms “a,” “an,” and “the” include plural references unless the context clearly dictates otherwise. Thus, for example, unless the context dictates otherwise, reference to “a projection” provides disclosure of embodiments in which only a single such projection is provided, as well as embodiments in which a plurality of such projections are provided.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

As used herein “or” should be understood to be an inclusive or unless context dictates otherwise. For example, when separating items in a list, “or” should be interpreted as being inclusive, i.e., the inclusion of at least one, but also including more than one, of a number or list of elements, and, optionally, additional unlisted items. In other aspects, the term “or” can refer to only a single element of a list of elements.

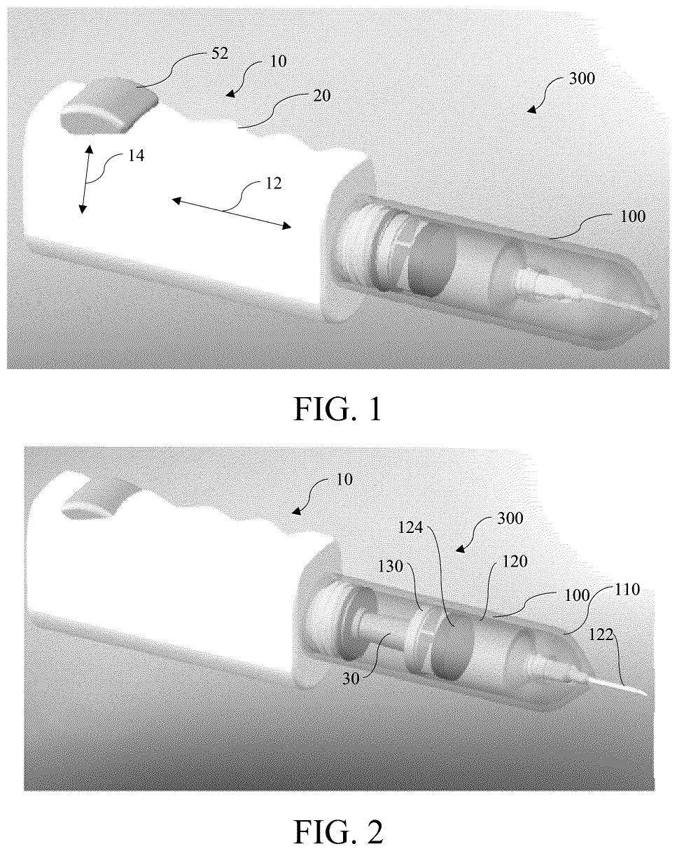

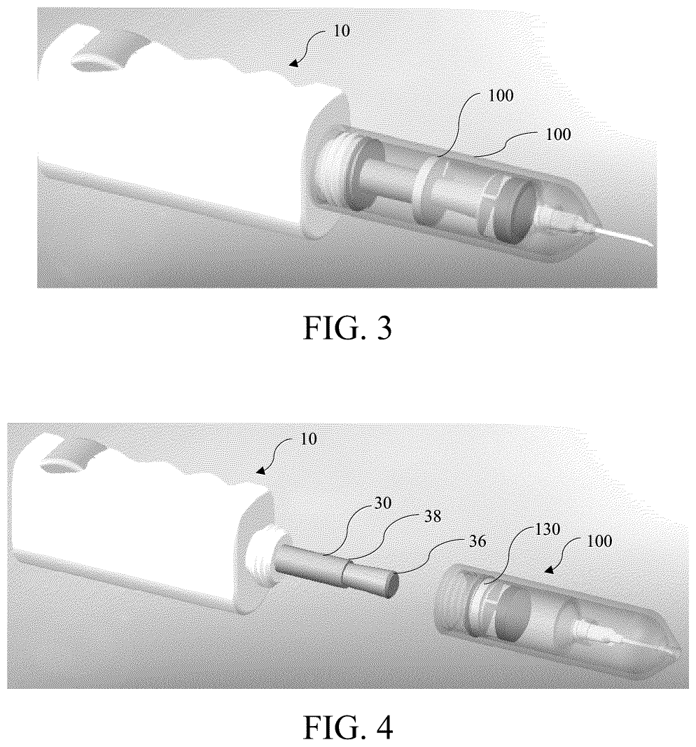

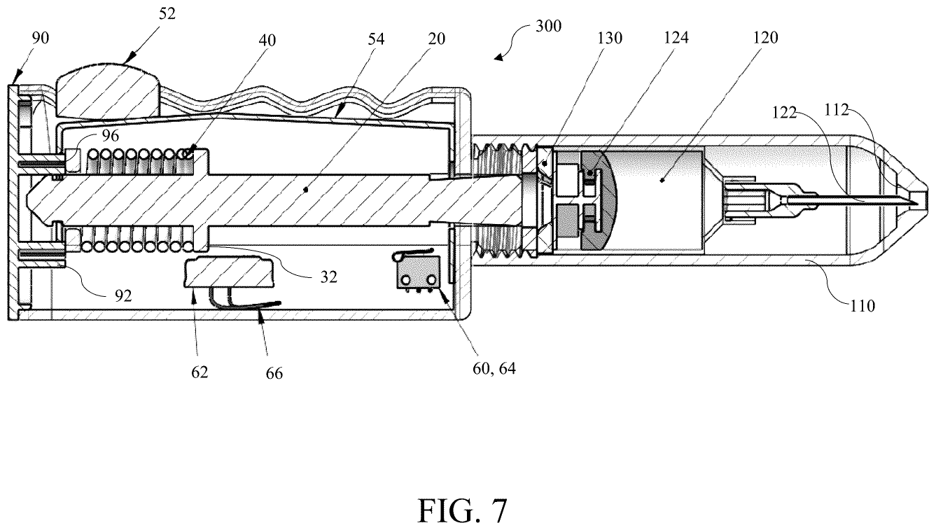

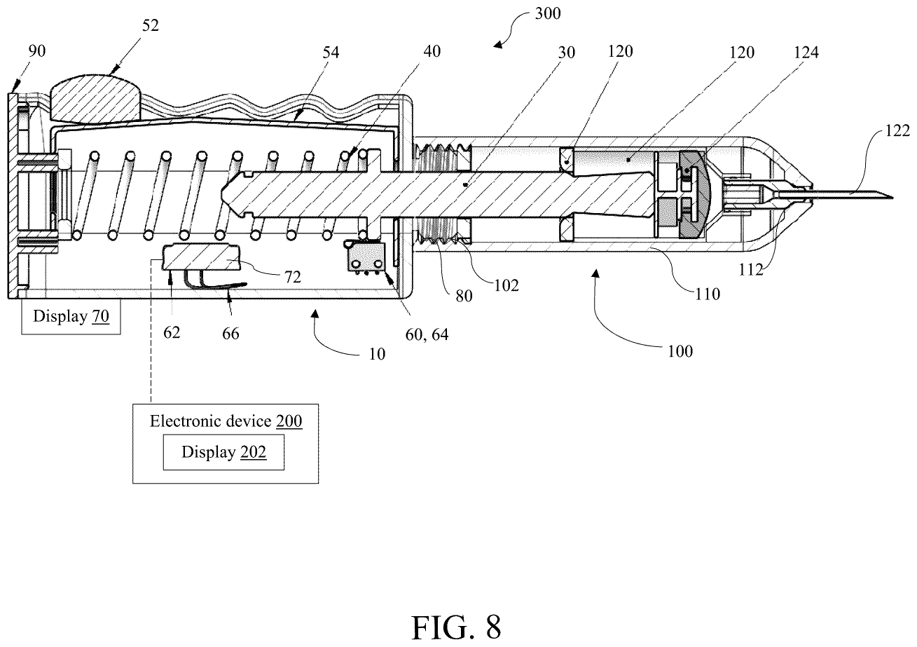

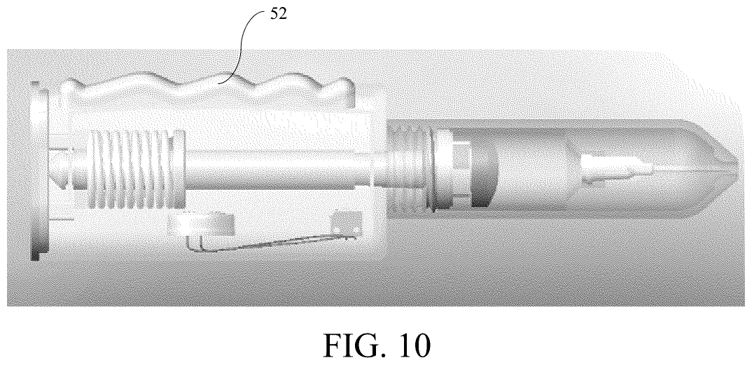

Disclosed herein, and with reference to FIGS. 1-4, 7, and 8, is an apparatus 10 configured to dispense medication from a cartridge 100. The apparatus 10 can comprise a housing 20 that is configured to couple to the cartridge 100. A piston 30 can be movable within the housing 20 along a path of travel from a first position (FIG. 7) to a second position (FIG. 8). The apparatus 10 can further comprise a spring 40 biasing the piston 30 toward the second position. A catch 50 can be configured to retain the piston in the first position. A trigger 52 can be configured to release the catch to permit the spring to move the piston to the second position. (Another embodiment of a trigger 52 is shown in FIG. 10.) A sensor 60 can be configured to detect movement of the piston 30 from the first position. The apparatus 10 can further comprise a controller 62 in electrical communication with the sensor 60 (e.g., via wires 66). The controller 62 can be configured to initiate a timer upon receiving a signal from the sensor indicative of movement of the piston from the first position.

In this way, the apparatus 10 can be configured to track a duration from medication dispensation. In some aspects, the timer can be a single duration associated with the sensor detecting movement of the piston from the first position. For example, the time can be a duration since the sensor 60 sensed movement of the piston 30 from the first position and, thus, the duration since medication dispensation.

In some aspects, the sensor 60 can be (or comprise) a switch 64 that is configured to switch during movement of the piston from the first position to the second position. For example, the switch 64 can be positioned within the housing 20 so that the piston 30 contacts the switch as the piston moves from the first position to the second position. Optionally, in these aspects, the piston 30 can comprise a radially extending flange 32. The flange 32 can be configured to contact the switch 64 during movement of the piston 30 from the first position to the second position. In other optional aspects, it is contemplated that the sensor 60 can be a proximity sensor or motion sensor as is known in the art, with the sensor configured to detect a position or motion of a portion of the piston such that movement from the first position can be detected.

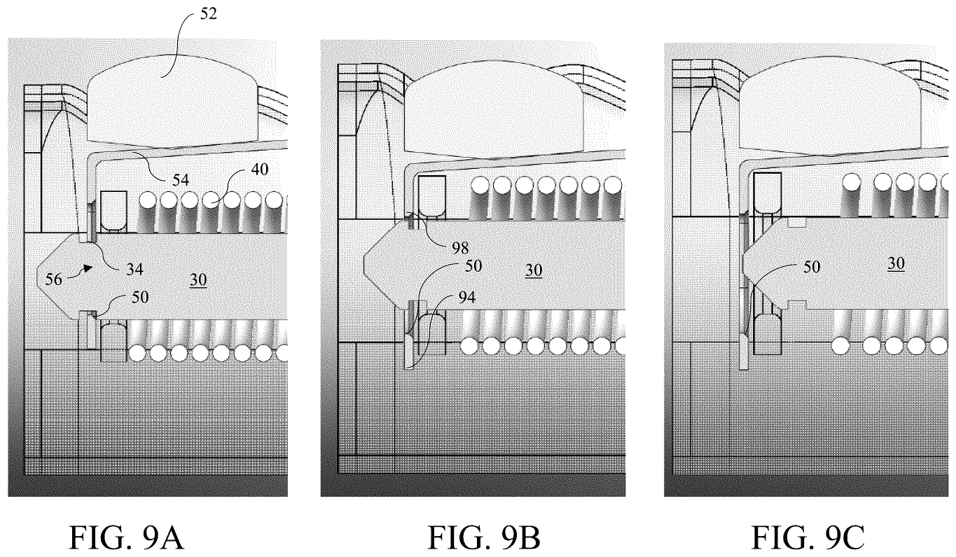

Referring to FIGS. 9A-9C, the piston 30 can define a notch 34 (e.g., an annular recess) that receives a portion of the catch 50 when the piston is in the first position. The catch 50 can be movable relative to the piston 30 and out of the notch 34 upon operation of the trigger 52. For example, the piston can be movable along a first axis 12, and the catch 50 can be movable along a second axis 14 that is transverse to the first axis. In exemplary aspects, the catch 50 can be a portion of a resilient arm 54 that deflects to move the catch 50 in and out of engagement with the piston 30. Accordingly, in some aspects, the catch 50 can be biased to a position in which the catch retains the piston 30 in the first position. In some aspects, the piston 30 can extend through an opening 56 in the resilient arm 54, and the catch 50 can comprise surfaces of the resilient arm 54 proximate to the opening 56 and that are configured to engage the notch 34. Although specific examples of the structure of the catch 50 are disclosed, it is contemplated that the catch 50 can have any suitable structure (e.g., a projection, a wedge, a latch, and the like) for engaging the piston 30 and being displaced by the trigger 52 to permit movement of the piston as disclosed herein. Similarly, although the trigger 52 is shown as being configured for being pressed to displace the catch 50 along axis 14 in a first direction, in other aspects, it is contemplated that the trigger 52 can be provided as a slide mechanism that is configured to displace the catch 50 along axis 12, or as a pull mechanism that is configured to displace the catch along axis 14 in a second direction opposite the first direction.

Referring to FIGS. 7, 8, and 9B, in some aspects, the apparatus 10 can comprise a safety cap 90 that inhibits movement of the catch 50 out of engagement with the piston 30. For example, as shown in FIG. 7, the safety cap 90 can comprise a first projection 92 that is positioned to engage a first surface 94 (FIG. 9B) of the catch 50 to inhibit movement thereof. In further aspects, the safety cap 90 can comprise a second projection 96 that is positioned to engage a second surface 98 (FIG. 9B) of the catch 50 to inhibit movement thereof. In exemplary aspects, the first surface 94 can be a distal end of the resilient arm 54. In further exemplary aspects, the second surface 96 can be an interior surface of the opening 56 in the resilient arm 54. The safety cap 90 can be removed prior to operation of the trigger 52 to permit use of the apparatus.

In some aspects, the apparatus 10 can further comprise a display 70 (shown schematically in FIG. 9) in communication with the controller. The display 70 can be configured to display a readout of the timer. The display 70 can comprise, for example, an LCD or LED screen.

In some aspects, the apparatus 10 can further comprise a communication module 72 that is configured to communicate with an electronic device 200 comprising a display 202 for displaying a readout of the timer. The electronic device 200 can be a smart device, such as, for example, a smartphone, tablet, and the like. In other aspects, the electronic device 200 can be a device specifically configured for use with the apparatus 10. In some aspects, the communication module 72 can be a wireless communication module (e.g., a BLUETOOTH or Wi-Fi enabled communication module). In some aspects, apparatus can be configured to communicate with the electronic device 200 via wired communication.

Accordingly, the apparatus 10 can comprise a display (e.g., LED/LCD screen) or otherwise communicate (e.g., via the communication module 72) when the apparatus was activated to deliver a first dose of medication. By knowing when the first dose enters the patient, subsequent dose timing can be determined. This can be advantageous for timing delivery of additional medication or other treatment following deliver of a medication such as NAC or epinephrine.

In some aspects, the apparatus 10 can be reusable. For example, in some aspects, the cartridge 100 can be replaceable. In some aspects, the housing can define at least one thread 80 that is configured to engage a corresponding at least one thread 102 of the cartridge 100. In some aspects, the housing can define a Luer lock fitting that engages a corresponding fitting of the cartridge 100.

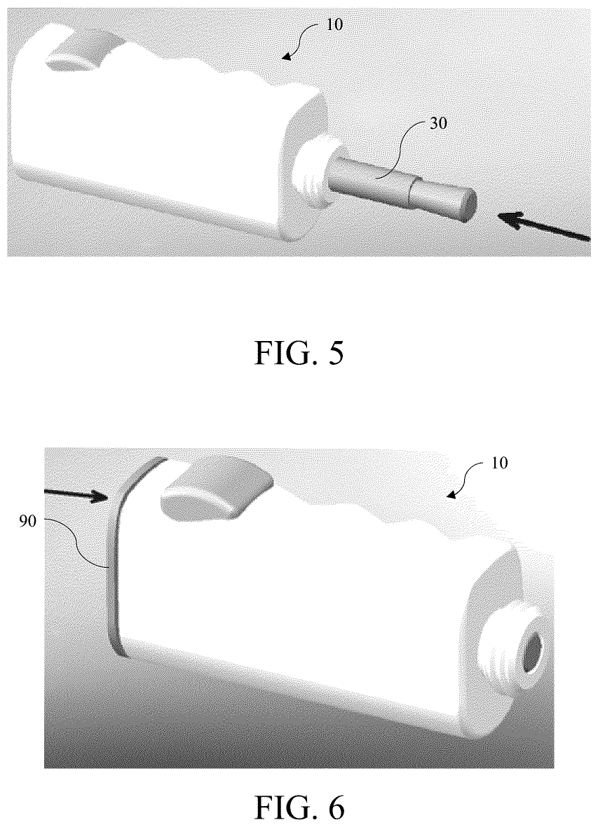

Referring to FIGS. 5-6, the piston can be pushed inwardly (arrow in FIG. 5) into the housing, thereby loading the spring 40 (FIG. 7). The safety cap 90 can then be inserted into the housing 20 (arrow in FIG. 6) for retaining the catch 50.

Referring to FIGS. 7-8, an autoinjector 300 can comprise the apparatus 10 with the cartridge 100 coupled to the housing. The cartridge 100 can comprise an enclosure 110 configured to couple to the housing 20 of the apparatus. A syringe 120 can be movable within the enclosure 110 from a stowed position (FIG. 1) to a deployed position (FIG. 2). The syringe 120 can contain a medication. The syringe 120 can comprise a needle 122. When the syringe is in the stowed position, the needle 122 can be within (e.g., entirely within) the enclosure 110, and when the syringe 120 is in the deployed position, the needle 122 can project from the enclosure 110. The syringe 120 can comprise a plunger 124 that is configured to engage the piston 30 as the piston 30 moves from the first position to the second position to effect flow of the medication through the needle 122.

In some aspects, the enclosure 110 can define a stop 112 that is configured to engage the syringe 120 when the syringe is at the deployed position. In some aspects, the stop 112 can be defined by a tapering, ridge, or other surface that inhibits further movement of the syringe 120. The cartridge 110 can comprise a ring 130 that is configured to engage the piston 30. The ring 130 can be movable within the enclosure 110 to push the syringe 120 toward the deployed position. The ring 130 and the piston 30 can cooperate to define a first threshold force above which the piston passes through the ring. Upon contact between the piston 30 and the ring 130, (before the syringe 120 reaches the stop) the piston 30 can be configured to apply to the ring 130 a force below the threshold force so that the piston pushes against the ring to push the syringe 120 to the second position until the syringe engages the stop. Upon the syringe contacting the stop, the syringe is configured to apply a force above the threshold force to drive the syringe through the ring and contact the plunger. In some aspects, the enclosure 110 can comprise a seal (e.g., a film) that is punctured as the needle 122 passes therethrough, thereby ensuring that the needle remains sterile prior to puncturing the skin of the patient. By keeping said needle sterile, the seal can help prevent infection.

It is contemplated that the ring 130 can prevent engagement between the piston 30 and the plunger 124 until the syringe is at the deployed position (with the needle deployed from the enclosure 110). In this way, the ring 130 can prevent medication dispensation until the needle has been deployed into the subject. The ring 130 and the piston 30 can cooperate to define the first threshold force based on a predetermined interference between the ring 130 and the piston 30 and the deformability of the ring 130 and the piston 30. For example, the ring 130 can be configured to deform (e.g., radially expand) to receive the piston 30 therethrough upon application of the first threshold force. In further aspects, the ring can comprise one or more projections (not shown) that extend radially inwardly and deflect upon application of the first threshold force. In some aspects, the ring 130 can be tapered in the movement direction of the piston 30 from the first position to the second position.

In some aspects, the medication can be sealed within the syringe (e.g., a sterile syringe). For example, the syringe 120 can comprise a seal that opens upon sufficient pressure applied thereto to permit release of the medication.

In some aspects, the piston 30 can have an end portion 36 (FIG. 4) having a first radial dimension (e.g., diameter). The piston 30 can have a smaller radial dimension spaced from the end portion 36 away from the end of the piston 30 that engages the plunger. The smaller radial dimension can permit the piston 30 to pass through the ring 130 without dragging. The piston 30 can have a second portion 38 (FIG. 4) having an increased radial dimension that is configured to engage the ring 130 to stop further movement of the piston 30.

Optionally, the cartridge 100 can comprise at least one bearing to guide movement of the piston 30.

In some aspects, the medication can comprise N-Acetylcysteine (NAC). In some aspects, the syringe can contain at least 1 mL, at least 2 mL, at least 3 mL, at least 4 mL, at least 5 mL, at least 6 mL, at least 7 mL, or at least 8 mL of medication. For example, the syringe can contain about 5 mL of medication (e.g., at least or about 5 mL of medication comprising NAC). In exemplary aspects, the syringe can contain from about 1 mL to about 20 mL (e.g., from about 3 mL to about 10 mL, or about 5 mL) of medication. More generally, the syringe can be any suitable size based on the medication contained therein.

The autoinjector can be used to deliver the medication (e.g., NAC) into a subject that has been suspected of having a heart attack, a stroke, a large dose of radiation exposure or other event causing increased free radical exposure, or loss of blood. For example, an individual on a battlefield who has lost an amount of blood to trauma can receive medication (e.g., NAC) from the autoinjector. The timer of the apparatus 10 can permit tracking of the time since dispensation. The treatment of the subject can be adjusted based on the time since dispensation. For example, once the subject is transported to a medical facility, a medical professional can use a readout of the timer of the apparatus to determine timing for delivery of additional medication or providing other treatment that depends upon the timing of the initial medication dispensation. More generally, the medication (e.g., NAC) can be advantageous for treating any medical conditions that increase reactive oxygen species/ischemia. Such medical conditions include, but are not limited to, heart attacks, strokes, battlefield blood loss related ischemic/reperfusion injuries, or high dose radiation exposure. In additional aspects, the medication used with the injector disclosed herein can be, for example, epinephrine.

Although the foregoing invention has been described in some detail by way of illustration and example for purposes of clarity of understanding, certain changes and modifications may be practiced within the scope of the appended claims.

What is claimed is:

1. An apparatus configured to dispense medication from a cartridge, the apparatus comprising:
a housing that is configured to couple to the cartridge;
a piston movable within the housing along a path of travel from a first position to a second position;
a spring biasing the piston toward the second position;
a catch that is configured to retain the piston in the first position;
a trigger that is configured to release the catch to permit the spring to move the piston to the second position;
a sensor that is configured to detect movement of the piston from the first position; and
a controller in electrical communication with the sensor, wherein the controller is configured to initiate a timer upon receiving a signal from the sensor indicative of movement of the piston from the first position.

2. The apparatus of claim 1, wherein the sensor is a switch that is configured to switch during movement of the piston from the first position to the second position.

3. The apparatus of claim 2, wherein the switch is positioned within the housing so that the piston contacts the switch as the piston moves from the first position to the second position.

4. The apparatus of claim 3, wherein the piston comprises a radially extending flange, wherein the flange is configured to contact the switch during movement of the piston from the first position to the second position.

5. The apparatus of claim 1, wherein the piston defines a notch that receives a portion of the catch when the piston is in the first position, wherein the catch is movable relative to the piston and out of the notch upon operation of the trigger.

6. The apparatus of claim 5, wherein the piston is movable along a first axis, wherein the catch is movable along a second axis that is transverse to the first axis.

7. The apparatus of claim 1, wherein the catch is biased to a position in which the catch retains the piston in the first position.

8. The apparatus of claim 1, further comprising a display in communication with the controller, wherein the display is configured to display a readout of the timer.

9. The apparatus of claim 1, further comprising a communication module that is configured to communicate with an electronic device comprising a display for displaying a readout of the timer.

10. The apparatus of claim 9, wherein the communication module is a wireless communication module.

11. The apparatus of claim 1, wherein the apparatus is reusable.

12. The apparatus of claim 1, wherein the housing defines at least one thread that is configured to engage a corresponding at least one thread of the cartridge.

13. The apparatus of claim 1, wherein the housing defines a Luer lock fitting.

14. The apparatus of claim 1, wherein the timer is a single duration associated with the sensor detecting movement of the piston from the first position.

15. An autoinjector comprising:
the apparatus as in claim 1, and
a cartridge coupled to the housing, the cartridge comprising:
an enclosure configured to couple to the housing;
a syringe movable within the enclosure from a stowed position to a deployed position, the syringe containing a medication, the syringe comprising:
a needle, wherein:
when the syringe is in the stowed position, the needle is within the enclosure, and
when the syringe is in the deployed position, the needle projects from the enclosure;
wherein the syringe comprises a plunger that is configured to engage the piston as the piston moves from the first position to the second position to effect flow of the medication through the needle.

16. The autoinjector of claim 15, wherein the enclosure defines a stop that is configured to engage the syringe when the syringe is at the deployed position, wherein the cartridge comprises a ring that is configured to engage the piston, wherein the ring is movable within the enclosure to push the syringe toward the deployed position, wherein the ring and the piston cooperate to define a first threshold force above which the piston passes through the ring, wherein upon contact between the piston and the ring, the piston is configured to apply to the ring a force below the threshold force so that the piston pushes against the ring to push the syringe to the second position until the syringe engages the stop, and wherein, upon the syringe contacting the stop, the syringe is configured to apply a force above the threshold force to drive the syringe through the ring and contact the plunger.

17. The autoinjector of claim 15, wherein the medication comprises N-Acetylcysteine (NAC).

18. A method of using the autoinjector as in claim 15, the method comprising:
injecting the medication into a subject, wherein the subject has been suspected as having a heart attack, a stroke, a large dose of radiation exposure, or loss of blood.

19. A cartridge configured to couple to the housing of the apparatus as in claim 1, the cartridge comprising:
an enclosure configured to couple to the housing;
a syringe movable within the enclosure from a stowed position to a deployed position, the syringe containing a medication, the syringe comprising:
a needle, wherein:
when the syringe is in the stowed position, the needle is within the enclosure, and
when the syringe is in the deployed position, the needle projects from the enclosure,
wherein the syringe comprises a plunger that is configured to engage a piston to effect flow of the medication through the needle, and
wherein the enclosure defines a stop that is configured to engage the syringe when the syringe is at the deployed position, wherein the cartridge comprises a ring that is configured to engage the piston, wherein the ring is movable within the enclosure to push the syringe toward the deployed position, wherein the ring and the piston cooperate to define a first threshold force above which the piston passes through the ring, wherein upon contact between the piston and the ring, the piston is configured to apply to the ring a force below the threshold force so that the piston pushes against the ring to push the syringe to the second position until the syringe engages the stop, and wherein, upon the syringe contacting the stop, the syringe is configured to apply a force above the threshold force to drive the syringe through the ring and contact the plunger.

20. An autoinjector comprising:
a housing;
a piston movable within the housing along a path of travel from a first position to a second position;
a spring biasing the piston toward the second position;
a catch that is configured to retain the piston in the first piston;
a trigger that is configured to release the catch to permit the spring to move the piston to the second position; and
a cartridge coupled to the housing, the cartridge comprising:
an enclosure configured to couple to the housing;
a syringe movable within the enclosure from a stowed position to a deployed position, the syringe containing a medication, the syringe comprising:
a needle, wherein:
when the syringe is in the stowed position, the needle is within the enclosure, and
when the syringe is in the deployed position, the needle projects from the enclosure,
wherein the syringe comprises a plunger that is configured to engage the piston as the piston moves from the first position to the second position to effect flow of the medication through the needle, and
wherein the enclosure defines a stop that is configured to engage the syringe when the syringe is at the deployed position, wherein the cartridge comprises a ring that is configured to engage the piston, wherein the ring is movable within the enclosure to push the syringe toward the deployed position, wherein the ring and the piston cooperate to define a first threshold force above which the piston passes through the ring, wherein upon contact between the piston and the ring, the piston is configured to apply to the ring a force below the threshold force so that the piston pushes against the ring to push the syringe to the second position until the syringe engages the stop, and wherein, upon the syringe contacting the stop, the syringe is configured to apply a force above the threshold force to drive the syringe through the ring and contact the plunger.
